(19) United States
(12) Patent Application Publication
(10) Pub. No.: US 2022/0119510 A1
(43) Pub. Date: Apr. 21, 2022

(54) ANTI-POLYETHYLENE GLYCOL (PEG) ANTIBODY MOUSE MODEL FOR RIGOROUS ASSESSMENT OF PEG-BASED THERAPIES: ADJUVANT-FREE INDUCTION MODEL

(71) Applicant: Northwestern University, Evanston, IL (US)

(72) Inventors: Evan A. Scott, Evanston, IL (US); Guillermo A. Ameer, Evanston, IL (US); Helena Freire Haddad, Evanston, IL (US); Jacqueline A. Burke, Evanston, IL (US)

(21) Appl. No.: 17/503,057

(22) Filed: Oct. 15, 2021

Related U.S. Application Data
(60) Provisional application No. 63/092,019, filed on Oct. 15, 2020.

(51) Int. Cl. C07K 16/18; A61K 47/60; A61K 47/02

(52) U.S. Cl. CPC C07K 16/18; A61K 2039/505; A61K 47/02; A61K 47/60

(57) ABSTRACT

The present invention provides methods for generating a mouse model that produces anti-polyethylene glycol (PEG) antibodies. Also provided are mice generated by said methods and methods of using these mice to screen PEG-containing products in vivo.

### CROSS-REFERENCE TO RELATED APPLICATIONS

This application claims priority to U.S. Provisional Application No. 63/092,019 filed on Oct. 15, 2020, the contents of which are incorporated by reference in their entireties.

### STATEMENT REGARDING FEDERALLY SPONSORED RESEARCH

This invention was made with government support under DGE-1842165 awarded by the National Science Foundation Graduate Research Fellowship. The government has certain rights in the invention.

### BACKGROUND

Poly(ethylene glycol) (PEG) is a non-toxic, hydrophilic polymer composed of ethylene oxide monomers that can be combined into linear or branched polymer chains with various molecular weight^{1,2}. Over the past 40 years, PEG has shown great potential to overcome rapid clearance, low solubility, and high immunogenicity associated with peptide, protein, and small molecule drug delivery, and has therefore gained the attention of drug companies and researchers³⁻⁵. PEG can be used as an excipient, a drug carrier, or as a coating agent⁶. Through a technique called PEGylation, PEG chains are covalently attached to the surface of a drug or material of choice^{3,7}. Each PEG polymer subunit associates with two to three water molecules; the hydrated polymer shields the therapeutic from immunogenic recognition by neutralizing antibodies and the degradative action of proteolytic enzymes^{5,8}. Additionally, PEGylation increases the hydrodynamic diameter and molecular weight of PEGylated moiety, thereby limiting renal clearance and increasing circulation time⁵. This is particularly useful for nanosized therapeutics, as glomerular filtration depends heavily on the size and molecular weight of a particle due to the structure and permeability of the glomerulus⁸. Hence, particles with a hydrodynamic diameter larger than 8 nm cannot be filtrated and eliminated by the kidneys⁸. Due to its unique properties, PEG has become the go-to biomaterial to enhance delivery of therapeutic molecules⁹. As of 2020, there were 21 PEGylated drugs approved by the FDA, and over 20 others in active clinical trials^{10,11}.

In addition to the use of PEG in therapeutics, the polymer is extensively used as a solvent and emulsifying agent in household products¹². PEG can be found in everyday products such as shampoo, moisturizer, makeup, and soaps, and in topological agents¹². The prevalence of PEG in products highly utilized by society has significantly increased in the past decades, with a growing variety of chain sizes, structures and functional groups used in common products¹².

Although it had been initially thought that PEG was non-immunogenic⁵, anti-PEG antibodies were first observed in rabbits following immunization with PEGylated ovalbumin¹³. One year later, anti-PEG antibodies were detected in the blood of donors without previous exposure to PEGylated therapeutics¹⁴. The development of anti-PEG antibodies in humans is associated with daily exposure to PEG-containing products⁶. A 2016 study found that 72% of people carry detectable concentrations of “pre-existing” anti-PEG antibodies without prior exposure to PEG-based drugs⁶. This study found that the average anti-PEG antibody immunoglobulin G (IgG) concentration was 52 ng/ml in the general population⁶.

Due to the presence of pre-existing anti-PEG antibodies, patients receiving treatment with a PEGylated drug can experience accelerated blood clearance, pharmacokinetic changes with multiple does, decreased therapeutic function due to decreased therapeutic circulation time, and anaphylaxis^{6,14}.

During the drug development process, PEG-containing drugs are assessed using animal models that have not been exposed to the PEG-based products that are common in daily human life. Thus, these animal models do not possess the anti-PEG antibodies that are found in humans. As a result, many drugs that do well in animal studies fail in clinical trials due to unexpected results (e.g., inefficacy, adverse reactions) caused by the presence of anti-PEG antibodies in human blood. For example, in a 2013 Phase 2b clinical trial of Pegnivocagin, a PEGylated RNA aptamer for the inhibition of coagulation factor Ixa, patients with pre-existing anti-PEG antibodies developed anaphylactic and skin reactions, resulting in the termination of the trial^{15,16}.

Failure of drugs in human clinical trials pose a great financial and health burden on society. Bringing a drug from development to the market is estimated to cost over $2.5 billion¹⁷. Risk of failure in clinical trials is high, averaging 95%¹⁸. Thus, eliminating drug candidates that will fail in clinical trials before investor money and human health are put at risk is vital for societyl^{17,19}.

Thus, there is a great need for an animal model that recapitulates the concentrations of anti-PEG antibodies found in human blood and can be used to assess novel therapeutics prior to human trials.

### SUMMARY

The present disclosure provides an animal model for studying PEG-product effects and methods of making and using.

In one aspect, the disclosure provides a method for generating an animal that produces anti-polyethylene glycol (PEG) antibodies, the method comprising: administering to the animal a composition that: (a) comprises PEG in an amount effective to induce production of anti-PEG antibodies; and (b) does not comprise an adjuvant. In some aspect, the animal is a mouse.

In another aspect, the disclosure provides an animal that produces anti-PEG antibodies generated by the method described herein.

In another aspect, the disclosure provides a method for screening a PEG-containing product in vivo, the method comprising administering the product to the animal described herein.

### BRIEF DESCRIPTION OF THE DRAWINGS

FIG. 1 shows the anti-PEG IgG response following a single PEG exposure. Healthy C57BL/6 mice were injected subcutaneously with methoxy poly(ethylene glycol) (mPEG) (black) or PEGylated gold nanoparticles (AuNPs) (gray) at a dose of 6.75 E-08 mol PEG/kg. PEG chains varied in molecular weight: 2, 5, 10 and 20 K. A control consisting of milliQ water was used for the mPEG-treated groups and non-PEGylated AuNPs were used as a control for the PEGylated AuNP-treated groups. Blood samples were collected 28 days after the initial injection, and analyzed via in-house ELISAs that detected antibodies specific to backbone of PEG with a molecular weight of 5, 10 or 20 K. Two-way ANOVA was performed to analyze the data with *=p<0.05, **=p<0.01, ****=p<0.0001. (n=10 mice/group).

FIG. 2 shows the anti-PEG IgG response in a cohort of mice received a second (“booster”) dose 14 days after the first injection.

FIG. 3 shows a comparison of the anti-PEG IgG response in mice that received a single dose of mPEG (A, left panel), two doses of mPEG (A, right panel), a single dose of PEGylated AuNPs (B, left panel), and two doses of PEGylated AuNPs (B, right panel).

### DETAILED DESCRIPTION

The present invention provides methods for generating a mouse model that produces anti-polyethylene glycol (PEG) antibodies. The method does not use an adjuvant, which may effect the immune response in the model. Also provided are mice generated by said methods and methods of using these mice to screen PEG-containing products in vivo. A variety of approaches to create anti-PEG antibody-producing animal models have been described in literature. Mouse models are ideal due to their commonality in research, low cost, and small size²⁰. However, none have successfully captured the state of pre-existing anti-PEG antibodies known to be present in the general population via the same immunological mechanism (PEG exposure) or at relevant concentrations. In previous models, small animals have been injected with PEGylated proteins and an adjuvant or PEGylated liposomes (which act as adjuvants) to induce antibody production²¹. Because these adjuvants also enhance the immune response²³, the immune response to the PEG therapeutic cannot be distinguished from the immune response to the adjuvant. Thus, the immune response in the human body in the absence of the adjuvant cannot be predicted from these models.

A final issue with existing mouse models is that they are unable to recapitulate the steady state concentration of anti-PEG antibodies for an extended duration of time to facilitate testing of PEG-based therapeutics²³. Ideally, steady state maintenance of multiple concentrations of anti-PEG antibodies by different cohorts of mice would be achieved. This would allow PEG-based therapeutics to be assessed in organisms with different blood concentrations of antibodies to account for variations in the general population. This would be impactful as it has been previously seen that some drugs are safe and effective for patients with low anti-PEG antibody concentrations but are extremely dangerous for patients with high concentrations.

To overcome the shortcomings of previous models, the present inventors have developed a robust mouse model that produces anti-PEG antibodies. The primary advantage offered by the anti-PEG antibody-producing mouse models of the present invention it that they are adjuvant-free. As a result, the immune response generated in these mice is more representative of the immune response that is stimulated by the PEG therapeutic itself. This model can be used to screen PEG-based therapeutics for pharmacokinetics, biodistribution, effective dosing, and immunogenic responses in the presence of anti-PEG antibodies. Thus, this mouse model can be used to reduce costs associated with clinical trials for drugs that would fail due to the presence of anti-PEG antibodies.

### Methods for Generating Mice that Produce Anti-PEG Antibodies

In a first aspect, the present invention provides methods for generating a mouse that produces anti-polyethylene glycol (PEG) antibodies. The methods comprise administering to the mouse a composition that: (a) comprises PEG in an amount effective to induce production of anti-PEG antibodies; and (b) does not comprise an adjuvant.

As used herein, the term “anti-polyethylene glycol (PEG) antibody” refers to an antibody (e.g., an IgM or IgG antibody) or fragment thereof that is capable of binding to PEG, including any variation in molecular weight, structure (e.g. linear vs. branched), chemical functionalization, and non-covalent or covalent conjugation (e.g. PEGylated proteins, PEG-coated surfaces of macro- or nanoscale surfaces, PEG hydrogels etc.).

In the present methods, a composition that comprises PEG but does not comprise an adjuvant is used to induce the production of anti-PEG antibodies in mice. Thus, these compositions are sometimes referred to herein as the “induction composition”. PEG is a non-toxic, hydrophilic polymer composed of ethylene oxide monomers that can be combined into linear or branched polymer chains. The induction composition may comprise PEG as the sole ingredient or it may comprise additional nonimmunogenic ingredients, such as a dilution agent (e.g., saline phosphate buffer, water). The PEG used in the induction composition can be of any molecular weight. In one embodiment, the antibodies produced are specific to all MW PEG above 550 g/mol. In the Examples, the inventors administered induction compositions comprising PEG chains that varied in molecular weight from 2 kDa to 20 kDa to mice. Thus, in some embodiments, the induction composition comprises PEG chains with a molecular weight of 2 kDaK, 5 kDa, 10 kDa, and/or 20 kDa.

In the Examples, the inventors injected mice with two different forms of PEG: (1) methoxy poly(ethylene glycol) (mPEG) in free polymer form and (2) PEG bound to gold nanoparticles (i.e., small gold particles with a diameter of 1 to 100 nm). They found that the PEGylated AuNPs induced a stronger anti-PEG antibody response than mPEG. Thus, in some embodiments, the induction composition comprises PEGylated organic (e.g., carbon-based) or inorganic (e.g., silica, silver, nickel, platinum, iron oxide, zinc oxide, gadolinium, silica and titanium dioxide, selenium, copper, gold (Au), palladium, etc.).

As used herein, the terms “administering” and “administration” refer to any method of providing a composition to a subject. Suitable methods for administrating the induction composition to the mouse include, without limitation, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, intradermal administration, intrathecal administration, and subcutaneous administration. Single or multiple administrations may be carried out. In the Examples, the inventors administered induction compositions to mice via subcutaneous injection or intravenous injection, however, any of the above-routes may be used. Thus, in some embodiments, the induction composition is administered to the mouse via subcutaneous injection or intravenous injection.

As used herein, the term “effective amount” refers to an amount that is effective to induce production of anti-PEG antibodies in the mouse. Anti-PEG antibody production may be detected using any suitable method known in the art including, without limitation, enzyme-linked immunosorbent assay (ELISA), western blot, immunohistochemistry, immunocytochemistry, immunoblotting, flow cytometry, and fluorescence-activated cell sorting (FACS). The effective amount will vary depending on factors such as the formulation and dosage of the induction composition, the method of administration, and the mouse (e.g., strain, age, weight) being used. In the Examples, the inventors were able to detect anti-PEG antibodies in mice following administration of compositions comprising a dose of from about 1×10⁻¹⁰ mg/kg to 10,000 mg/kg, alternatively about 1×10⁻⁸ mg/kg to 100 mg/kg. In some embodiments, the composition is administered in a dose of 6.75×10⁻⁸ mol PEG/kg.

The average anti-PEG antibody immunoglobulin G (IgG) concentration in the general human population is 52 ng/ml⁶. Thus, in some embodiments, the “target concentration” of anti-PEG antibodies in the mouse is between 1 ng/ml to about 1000 mg/ml, for example, about 40 ng/ml and 100 mg/ml, alternatively about 40 ng/ml to about 1 mg/ml, alternatively from about 44 ng/ml and 65 ng/ml.

In the methods of the present invention, the induction composition may be administered to the mouse multiple times. For example, the composition may be administered to the mouse 2, 3, 4, 5, 6, 7, 8, 9, 10, or more times. In some embodiments, the mouse model receives one or more booster 3 days, 7 days, or 14 days after the initial administration. In the Examples, one cohort of mice received a second (“booster”) dose of the PEG-containing composition 14 days after the first injection. Thus, in some embodiments, the composition is administered to the mouse at least twice. In particular embodiments, a second dose of the composition is administered 14 days after the first administration. In some embodiments, booster doses are administered regularly to the mouse to maintain the antibody concentration for the lifetime of the animal. In some embodiments, the booster doses maintain the anti-PEG antibody concentration within 10% of a target concentration. In some embodiments, the booster injections comprise a lower dose of PEG compared to the initial dose. For example, in some embodiments, the booster dose is equal to half of or one-fourth of the initial loading dose. One skilled in the art will be able to calculate a proper booster dose to maintain the levels of anti-PEG antibodies within the mouse model.

The time between initial dose of PEG and the time in which experimentation on the mice can begin is dependent on various factors including the initial dose, the target anti-PEG antibody concentration, and the acceptable range of anti-PEG antibody concentrations around that target.

For example, in some embodiments, the time between the initial administration and experimentation is from 5 days to 50 days, from 10 days to 45 days, or from 25 days to 40 days. In the Examples, anti-PEG antibodies were detected via ELISA in blood samples collected 28 days after the initial injection with a dose of 6.75×10⁻⁸ mol PEG/kg. Thus, in some embodiments the method generates a mouse that produces anti-PEG antibodies within a few days (e.g., 2-5 days) to any number of days in the mouse life cycle. How quickly the mice generate immune responses will vary depending on many experimental factors. In some embodiments, the antibody responses occurs within about 14 days, but this can differ depending on age, health, and the type of immunostimulation. This mouse model could also be used to investigate the generation of antibodies slowly over long periods of minor PEG exposure, so the model described herein can used to assess longer periods. The model can also be used to assess the duration of antibody responses as well as treatments to inhibit antibody generation, so the duration could last almost any period of time from a few days to permanent antibody response. An antibody response in mice may be seen from a few (5-7) days until any point in the mouse lifetime. The antibodies produced can persist for at least 1 day and potentially permanently for the lifetime of the mouse.

### Animal Model

In a second aspect, the present invention provides an animal that produce anti-PEG antibodies. The animal of the present invention are generated using the methods described above. The animal may be any research animal, for example, mouse, rat, rabbit, pig, NHP, dog, cat, hamster, guinea pigs, birds, fish, frog, camel, cow, horse, llama, and non-human primate. In the preferred embodiment, the research animal is a mouse or rat. An exemplary animal model is the mouse model demonstrated in the Examples.

Any strain of mouse may be used with the present invention. Suitable mouse strains include, without limitation, C57BL/6, BALB/c, CD-1, SCID, and A/J. In some embodiments, the mice are C57BL/6J mice. Mice of various strains are available from commercial suppliers, such as Jackson Laboratories, Charles River Laboratories, International, Laboratory Corporation of America Holdings, among others.

### Methods for Screening PEG-Containing Products in the Animal

In a third aspect, the present invention provides methods for screening a PEG-containing product in vivo. The methods comprise administering the product to an anti-PEG antibody-producing animal model described herein.

As used herein, the term “screening” refers to a process in which the characteristics or effects of a PEG-containing product are observed in vivo in the presence anti-PEG antibodies.

The term screening can encompass immune responses and reactions (e.g., immunogenicity), along with other side-effects of PEG-containing products on the animal model.

In some embodiments, the methods further comprise comparing the characteristics of the PEG-containing product (e.g., its half-life) in the anti-PEG antibody-producing animal to its characteristics in a control animal. Likewise, in some embodiments, the methods further comprise comparing the effects of the PEG-containing product (e.g., antibody production) in the anti-PEG antibody-producing animal to its effects in a control animal. As used herein, the term “control animal” refers to a comparable animal that does not contain anti-PEG antibodies. For example, it may be a mouse of the same strain as the mouse model described herein that has not been dosed with the PEG-containing composition.

PEG can induce immunological responses in patients due to the presence of pre-existing anti-PEG antibodies in their blood. This is problematic because an immune response to the product can limit its efficacy, especially when multiple doses of a product are needed. Thus, in some embodiments, the screen is used to determine the immunogenicity of the product in the presence of anti-PEG antibodies. As used herein, the term “immunogenicity” refers to the ability of a product to stimulate an immune response in a subject. Immunogenicity can be assessed by measuring the antibodies generated against the product. Antibody production may be measured using many standard techniques including, without limitation, ELISA, western blot, immunohistochemistry, immunocytochemistry, immunoblotting, flow cytometry, and fluorescence-activated cell sorting (FACS), mes-scale discovery (MSD), electrochemiluminescence (ECL), gyrolab, among others.

One consequence of PEG's immunogenicity is that human patients can experience an allergic reaction in response to a PEG-containing product. Thus, in some embodiments, the screen is used to identify a potential adverse reaction to the product that occurs in the presence of anti-PEG antibodies. As used herein, the term “adverse reaction” refers to any unexpected or dangerous reaction to a product. The onset of the adverse reaction may be sudden or develop over time. Exemplary adverse reactions to PEG include, without limitation anaphylaxis and skin reactions. Methods for detecting such reactions are known in the art. For example, IgG1 anaphylactic antibodies may be detected as an indication of anaphylaxis, and the skin of the animal or mouse can be visually observed to detect an allergic reaction. Most commonly the PEG-containing drug is more readily cleared from the body, thus it does not have its intended effect. In some embodiments, the adverse reaction is a complement activation-related pseudoallergy (CARPA). In some embodiments, the response is death. Anaphylaxis can be measured by methods known in the art, for example, by FcERI on mast cells, basophils, macrophages and Langerhans cells, FcyRIIb on mast cells, FcyRIIIon macrophages, generally FcyRIIA, FcyRIIIA, FC\cyRIIIC, production of histamine by mast cells, production of platelet activating factor by macrophages, and binding of IgE to FcyRIIb and FcyRIII.

A second consequence of the immunogenicity of PEG is that human patients receiving treatment with a PEGylated drug can experience accelerated blood clearance, pharmacokinetic changes, and decreased therapeutic function. Thus, in other embodiments, the screen may be used to determine the effective dose, pharmacokinetics, and/or biodistribution of a PEG-containing drug (e.g., a drug, adjuvant, nanoparticle, or nanocarrier) in the presence of anti-PEG antibodies. These characteristics can be determined using conventional methods such as dose response assays, pharmacokinetic assays, and biodistribution studies.

The screening methods described herein may also be used for understanding how variations in anti-PEG antibody concentrations within human populations impact the effective dose, pharmacokinetics, biodistribution, immunogenicity, and potential adverse reactions to a PEG-containing product. To this end, mice that produce varying levels of anti-PEG antibodies can be generated (e.g., by varying the dosage of the PEG-containing composition or the method of administration used to induce the antibodies). To generate mice with different levels of antibodies, one can alter one or more of the following conditions: alter dose (e.g., amount of PEG administerd), number of doses, frequency of doses, molecular weight of the PEG used to induce the antibodies, route of administration, formulation of the PEG, steric presentation of the PEG (free vs. nanoparticle bound) and combinations thereof.

As used herein, the term “PEG-containing product” is used to refer to a product that contains PEG. The methods of the present invention are designed to test the effects and characteristics of a PEG-containing product following administration to a subject.

PEG is widely used in drug delivery. For example, covalent attachment of PEG to a drug can be used to mask the drug from the subject's immune system, increase its hydrodynamic size (which prolongs its circulatory time by reducing renal clearance), and provide water solubility to hydrophobic drugs. Thus, in some embodiments, the PEG-containing product is a drug. Examples of FDA-approved PEGylated drugs include, without limitation, Skytrofa (Ascendis), Empaveli (Apellis), Nyvepria (Pfizer Inc.), Esperoct (Novo Nordisk), Ziextenzo (Sandoz), Udenyca (Coherus Biosciences), Palynziq (BioMarin Pharmaceutical), Revcovi (Leadiant Bioscience), Fulphila (Mylan GmbH), Asparlas (Servier Pharma), Jivi (Bayer Healthcare), Rebinyn (Novo Nordisk), Adynovate (Baxalta), Plegridy (Biogen), Omontys (Takeda), Sylatron (Merck), Krystexxa (Horizon Pharma), Cimzia (UCB), Mircera (Roche), Macugen (Pfizer), Somavert (Pfizer), Neulasta (Amgen), Pegasys (Roche), Pegintron (Schering), Oncaspar (Enzon), Adagen (Enzon), Movantik (AstraZeneca), Asclera (Chemische Fabrik Kreussler), and Doxil (Schering).

PEG is also used to coat nanoparticles/nanocarriers (e.g., those used for drug delivery), and medical devices because it creates a steric barrier that prevents opsonization and prolongs retention time in the body. Thus, in some embodiments, the PEG-containing product is a

PEGylated nanoparticle, nanocarrier, or medical device.

Due to its immunogenicity, PEG may also be used as an adjuvant, i.e., a substance that enhances the immune response to a vaccine. PEGylated compounds and PEG-based nanoparticles/nanocarriers can all be used as adjuvants. Thus, in some embodiments, the PEG-containing product is an adjuvant.

PEG is commonly used as a food additive because it helps to preserve moisture and to dissolve colors and flavors. Thus, in some embodiments, PEG-containing product is a food product, i.e., a substance that can be used or prepared for use as food.

PEG is also widely used in household products, such as skin care products, cosmetics, baby wipes, and cleaners. In such products, PEG is often used as a thickener, softener, moisture-carrying agent, penetration enhancer, or surfactant. Thus, in some embodiments, the PEG- containing product is a personal care product (e.g., a skin moisturizer, perfume, lipstick, fingernail polish, makeup, shampoo, hair color, toothpaste, deodorant) or a cleaning product (e.g., a detergent or other household cleaner).

In the methods of the present invention, the PEG-containing product may be administered to the animal model using any suitable method. Methods of administration include, without limitation, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, intradermal administration, intrathecal administration, and subcutaneous administration. For example, in embodiments in which the PEG-containing product is a PEGylated device, the device may be administered topically, orally or via implantation within the animal model. In embodiments in which the PEG-containing product is a food product, the product may be administered orally or fed to the animal model. In embodiments in which the PEG-containing product is a personal care product, the product may be administered topically. In embodiments in which the PEG-containing product is a cleaning product, the product may be administered topically, via skin contact, or via contact to a mucosal membrane.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. For example, with regard to sequences “consisting of” refers to the sequence listed in the SEQ ID NO. and does refer to larger sequences that may contain the SEQ ID as a portion thereof.

All publications, patent applications, patents, and other references mentioned herein are incorporated by reference in their entirety. In the case of conflict, the present specification, including definitions, will control.

Other features and advantages of the invention will be apparent from the description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting.

### EXAMPLES

### Example 1

In the following example, the inventors describe the generation of their anti-PEG antibody animal model, particularly a mouse model. By injecting mice with PEG chains of a variety molecular weights, in both free and nanoparticle form, they have better defined the conditions in which anti-PEG antibodies are formed.

### Materials and Methods

### Animals

8 to 12-week-old, male C57BL/6J mice were purchased from Jackson Labs. All animal protocols were approved by an Institutional Animal Care and Use Committee (IACUC).

### ELISA

An in-house enzyme-linked immunosorbent assay (ELISA) was developed to quantify the concentration of anti-PEG IgG in mouse blood samples. Amine-coated 96-well plates (Life Science) were incubated with a 4 mM N-hydroxysuccinimide-mPEG 5 kDa, 10 kDa, or 20 kDa (Nanocs) solution for 45 minutes at 37° C. The wells were washed with PEG Wash Buffer (Life Diagnostics) and blotted three times. Plates were blocked overnight with 125 ul of PEG blocking buffer (Life Diagnostics, Inc.) per well and washing was repeated. Standards of known anti-PEG IgG concentration (0-200 ng/ml) were made by diluting a backbone-specific anti-PEG monoclonal antibody, clone 1D9-6 (Life Diagnostics, Inc.), in whole mouse blood. Solutions of unknown and known concentrations were diluted by half with PEG Blocking Buffer (Life Diagnostics, Inc.). 100 μl of each solution was plated and incubated at room temperature for 2 hours with mild agitation. The wells were then washed and blotted five times. Goat anti-mouse IgG (H+L) horseradish peroxidase (HRP) secondary antibody (Sigma) was diluted in PEG Blocking Buffer in a 1:3000 dilution. 50 μl of the resulting solution was plated and incubated at room temperature for 45 minutes. The wells were then washed and blotted five times. Finally, the plate was incubated with 50 μl tetramethylbenzidine (Sigma), and after 15 minutes, 50 μl of 0.2 M sulfuric acid (Fisher) was added to each well. The absorbance of each well was read at 450 nm on a Cytation 5 (BioTek). Absorbance was related to concentration to determine concentration of anti-PEG IgG of samples with unknown concentrations.

### Induction

Mice were subcutaneously injected with 2K, 5K, 10K, or 20K molecular weight mPEG, in free polymer form (Nanocs) or bound to PEGylated gold nanoparticles (AuNPs; Luna Nanotech). AuNPs that had not been PEGylated and milliQ water were used for controls. One cohort of mice was subjected to a boost of the same concentration and modality of PEG. After 28 days, animals were sacrificed and underwent blood collection through cardiac puncture.

### Results

Healthy C57BL/6 mice were injected subcutaneously with methoxy poly(ethylene glycol) (mPEG) or PEGylated gold nanoparticles (AuNPs) at a dose of 9.48 mol PEG per kg body weight. (Note: This is the same amount of PEG that is present in one dose of Doxil®, as scaled for mouse body weight. Doxil® is a chemotherapeutic drug containing doxorubicin encapsulated in a PEGylated liposome. Calculations are based on the FDA approved dosage of 50 mg doxorubicin per m² body surface area for the treatment of ovarian cancer. The FDA's human equivalent dosage (HED) was used to calculate dosage between species. A Km of 3 is used to convert an animal dosage (mg/kg) to a human dosage (mg/m2). Doxil contains 2 mg doxorubicin per ml and 3.19 mg mPEG2000-DSPE per ml. mPEG2000-DSPE has a MW of 2805.5 g/mol. Doxil is indicated for ovarian cancer at a dose of 50 mg per m² body surface area. Assuming the average women has a body surface area of 1.6 m². Thus, this person would receive a dose of 80 mg doxorubicin, which is accompanied by 127.6 mg or 0.00004548 mol mPEG2000-DSPE. Thus, this person is given 0.00002843 mol per m². The FDAs's Km for converting an animal dose (mg/kg) to a human equivalent dose (HED; mg/m²) is 3 for mice. Thus, to determine the mouse dose from the HED, we need to divide by 3. Mice should receive 0.00000948 mol per kg (https://www.fda.gov/regulatory-information/search-fda-guidance-documents/estimating-maximum-safe-starting-dose-initial-clinical-trials-therapeutics-adult-healthy-volunteers). PEG chains varied in molecular weight: 2, 5, 10, and 20 K. A control consisting of milliQ water was used for the mPEG-treated groups and a control consisting of non-PEGylated AuNPs was used for the PEGylated AuNP-treated groups. One cohort of mice received a second (“booster”) dose 14 days after the first injection. Blood samples were collected 28 days after the initial injection and were analyzed via in-house ELISAs that detected antibodies specific to backbone of PEG with a molecular weight of 5, 10, or 20 K (n=10 mice/group). Two-way ANOVA was performed to analyze the data with *=p<0.05, **=p<0.01, ****=p<0.0001. (n=10 mice/group).

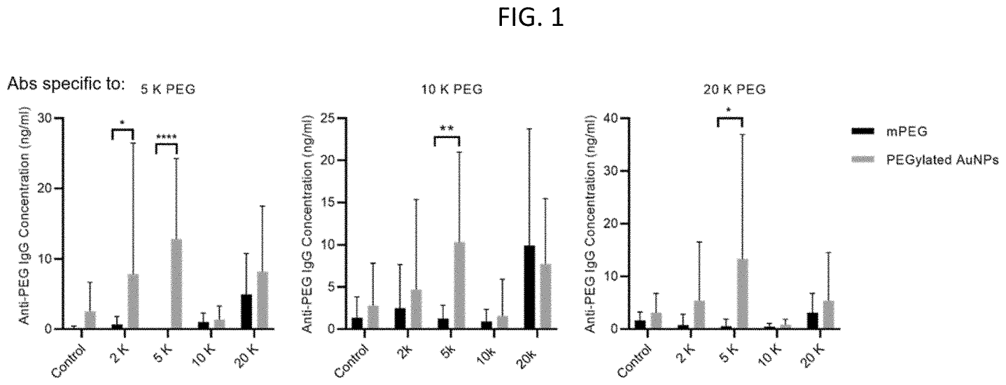

The results of this experiment show that following a single exposure of PEG MW 5 kDa, PEGylated AuNPs induced a stronger anti-PEG antibody response than mPEG (FIG. 1). This trend is nonspecific to PEG MW, such that the antibodies that are produced bind to all PEG MW that were assessed. This indicates that steric presentation of the PEG is critical to induce a response. We hypothesize that because the free form mPEG lacks the steric hinderance to prevent from interacting with itself, it effectively “balls up,” preventing presentation to and recognition by cells. Alternatively, the presentation of the PEG on the AuNPs provides the steric hinderance to prevent the PEG from interacting with itself. As a result, PEGylated AuNPs allow for PEG presentation and recognition by cells. Thus, antibody production occurs.

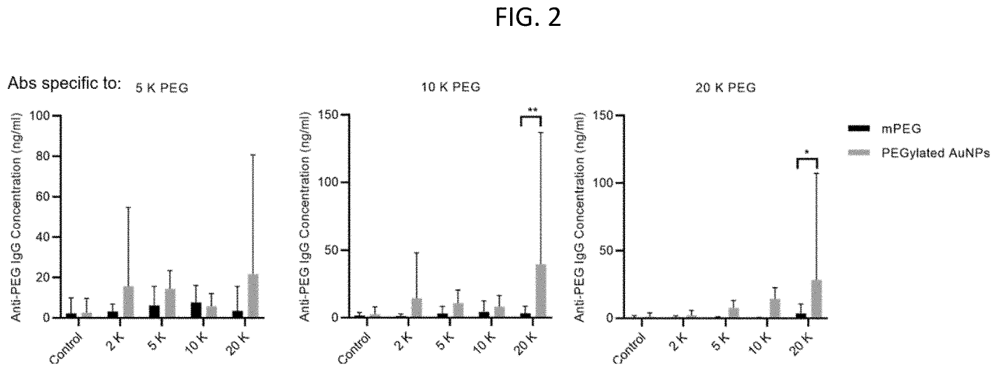

Furthermore, a single exposure to AuNPs PEGylated with PEG 5 K induces the greatest antibody response. Again, the antibodies produced are nonspecific in regard to the MW of the PEG that they bind. Thus, the MW of the PEG used for induction is important. We hypothesis that steric hindrance may also in regard to PEG MW and anti-PEG antibody production. If PEG MW is too low, the length of the PEG chain is too short and a “brush-like” conformal coating occurs inhibiting interaction with cells. Alternatively, if the PEG length is too long, the PEG will interact with itself, as opposed to presenting to cells. This type of coating is known as a “mushroom” conformation. Density of the PEG grafting also plays a role in the steric presentation of the PEG. With the two-exposure protocol, induction using AuNPs PEGylated with PEG MW 20 kDa resulted in increased antibody production (FIG. 2). At the 20 kDa PEG MW, AuNPs showed significant increases in antibodies production reactive to 10 kDa and 20 kDa PEG. Induction with AuNPs PEGylated with PEG MW 20 kDa produced the highest concentration of induced antibodies over all other MW. These antibodies were specific to all assessed PEG MWs. Once more, steric presentation and PEG MW were implicated as important factors for induction.

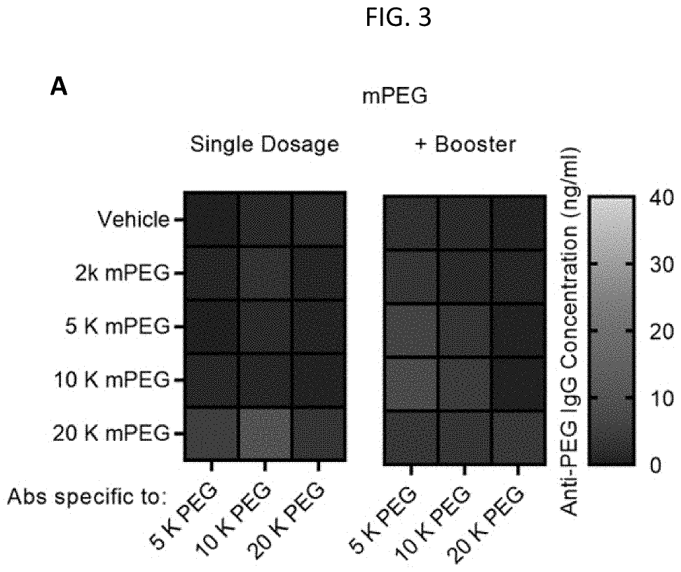

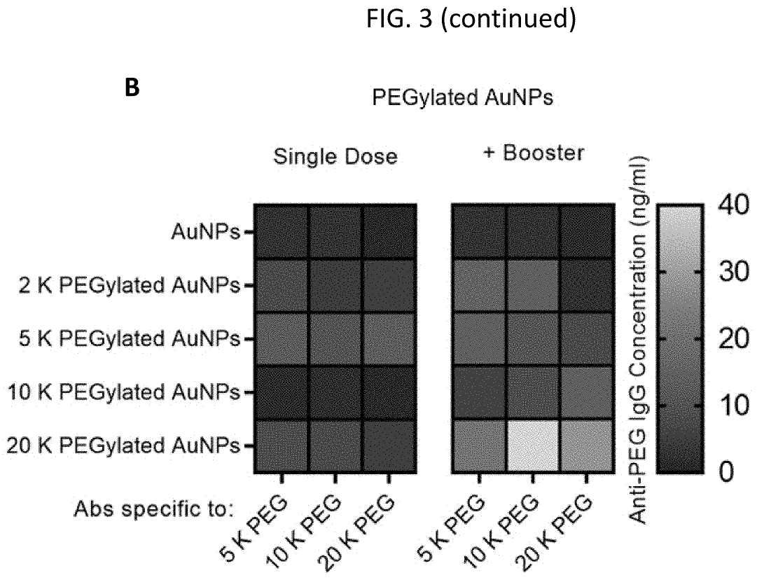

Two exposures of PEG resulted in significantly higher anti-PEG antibody concentration as compared to a single exposure (FIG. 3). Importantly, two doses of 20 kDa PEGylated AuNPs most reliably induced anti-PEG antibodies. The produced antibodies were specific to all assessed MW of PEG, with preference for PEG MW 10K. Given that the two dose 20 kDa PEGylated AuNP protocol produced the most robust response, it is recommended that this protocol be used for antibody induction.

### REFERENCES

1 Fruijtier-Polloth, C. Safety assessment on polyethylene glycols (PEGs) and their derivatives as used in cosmetic products. Toxicology 214, 1-38, doi:10.1016/j.tox.2005.06.001 (2005).
2 Delgado, C., Francis, G. E. & Fisher, D. The uses and properties of PEG-linked proteins. Crit Rev Ther Drug Carrier Syst 9, 249-304 (1992).
3 Harris, J. M. & Chess, R. B. Effect of pegylation on pharmaceuticals. Nat Rev Drug Discov 2, 214-221, doi:10.1038/nrd1033 (2003).
4 Veronese, F. M. & Pasut, G. PEGylation, successful approach to drug delivery. Drug Discov Today 10, 1451-1458, doi:10.1016/51359-6446(05)03575-0 (2005).
5 Abuchowski, A., van Es, T., Palczuk, N. C. & Davis, F. F. Alteration of immunological properties of bovine serum albumin by covalent attachment of polyethylene glycol. J Biol Chem 252, 3578-3581 (1977).
6 Yang, Q. et al. Analysis of Pre-existing IgG and IgM Antibodies against Polyethylene Glycol (PEG) in the General Population. Anal Chem 88, 11804-11812, doi:10.1021/acs.analchem.6b03437 (2016).
7 Harris, J. M., Martin, N. E. & Modi, M. Pegylation: a novel process for modifying pharmacokinetics. Clin Pharmacokinet 40, 539-551, doi:10.2165/00003088-200140070-00005 (2001).
8 Deen, W. M., Lazzara, M. J. & Myers, B. D. Structural determinants of glomerular permeability. Am J Physiol Renal Physiol 281, F579-596, doi:10.1152/ajprenal.2001.281.4.F579 (2001).
9 Hoang Thi, T. T. et al. The Importance of Poly(ethylene glycol) Alternatives for Overcoming PEG Immunogenicity in Drug Delivery and Bioconjugation. Polymers (Basel) 12, doi:10.3390/polym12020298 (2020).
10 Swierczewska, M., Lee, K. C. & Lee, S. What is the future of PEGylated therapies? Expert Opin Emerg Drugs 20, 531-536, doi:10.1517/14728214.2015.1113254 (2015).
11 Ramos-de-la-Peña, A. M. & Aguilar, O. Progress and Challenges in PEGylated Proteins Downstream Processing: A Review of the Last 8 Years. Int. J. Pept. Res. Ther. 26, 333-348, doi:10.1007/s10989-019-09840-4. (2020).
12 Jang, H. J., Shin, C. Y. & Kim, K. B. Safety Evaluation of Polyethylene Glycol (PEG) Compounds for Cosmetic Use. Toxicol Res 31, 105-136, doi:10.5487/TR.2015.31.2.105 (2015).
13 Richter, A. W. & Akerblom, E. Antibodies against polyethylene glycol produced in animals by immunization with monomethoxy polyethylene glycol modified proteins. Int Arch Allergy Appl Immunol 70, 124-131, doi:10.1159/000233309 (1983).
14 Richter, A. W. & Akerblom, E. Polyethylene glycol reactive antibodies in man: titer distribution in allergic patients treated with monomethoxy polyethylene glycol modified allergens or placebo, and in healthy blood donors. Int Arch Allergy Appl Immunol 74, 36-39, doi:10.1159/000233512 (1984).
15 Ganson, N. J. et al. Pre-existing anti-polyethylene glycol antibody linked to first-exposure allergic reactions to pegnivacogin, a PEGylated RNA aptamer. J Allergy Clin Immunol 137, 1610-1613 e1617, doi:10.1016/j.jaci.2015.10.034 (2016).
16 Povsic, T. J. et al. A Phase 2, randomized, partially blinded, active-controlled study assessing the efficacy and safety of variable anticoagulation reversal using the REG1 system in patients with acute coronary syndromes: results of the RADAR trial. Eur Heart J34, 2481-2489, doi:10.1093/eurheartj/ehs232 (2013).
17 DiMasi, J. A., Grabowski, H. G. & Hansen, R. W. The cost of drug development. N Engl J Med 372, 1972, doi:10.1056/NEJMc1504317 (2015).
18 Hay, M., Thomas, D. W., Craighead, J. L., Economides, C. & Rosenthal, J. Clinical development success rates for investigational drugs. Nat Biotechnol 32, 40-51, doi:10.1038/nbt.2786 (2014).
19 Aad, G. et al. Search for Magnetic Monopoles and Stable High-Electric-Charge Objects in 13 Tev Proton-Proton Collisions with the ATLAS Detector. Phys Rev Lett 124, 031802, doi:10.1103/PhysRevLett.124.031802 (2020).
20 Vandamme, T. F. Use of rodents as models of human diseases. J Pharm Bioallied Sci 6, 2-9, doi:10.4103/0975-7406.124301 (2014).
21 Ozmen, L. et al. Inhibition of thrombin abrogates the instant blood-mediated inflammatory reaction triggered by isolated human islets: possible application of the thrombin inhibitor melagatran in clinical islet transplantation. Diabetes 51, 1779-1784, doi:10.2337/diabetes.51.6.1779 (2002).
22 Ganson, N. J., Kelly, S. J., Scarlett, E., Sundy, J. S. & Hershfield, M. S. Control of hyperuricemia in subjects with refractory gout, and induction of antibody against poly(ethylene glycol) (PEG), in a phase I trial of subcutaneous PEGylated urate oxidase.

Arthritis Res Ther 8, R12, doi:10.1186/ar1861 (2006).
23 Hsieh, Y. C. et al. Pre-existing anti-polyethylene glycol antibody reduces the therapeutic efficacy and pharmacokinetics of PEGylated liposomes.

Theranostics 8, 3164-3175, doi:10.7150/thno.22164 (2018).

What is claimed:

1. A method for generating an animal that produces anti-polyethylene glycol (PEG) antibodies, the method comprising: administering to the animal a composition that:
a) comprises PEG in an amount effective to induce production of anti-PEG antibodies; and
b) does not comprise an adjuvant.

2. The method of claim 1, wherein the composition comprises PEGylated nanoparticles.

3. The method of claim 2, the composition comprises PEGylated carbon-based or silica, silver, nickel, platinum, iron oxide, zinc oxide, gadolinium, silica and titanium dioxide, selenium, copper, gold (Au), or palladium nanoparticles.

4. The method of claim 3, wherein the particles are gold nanoparticles (AuNPs).

5. The method of claim 1, wherein the composition comprises PEG chains with a molecular weight of 2 kDa, 5 kDa, 10 kDa, and/or 20 kDa.

6. The method of claim 1, wherein the composition is administered to the animal via subcutaneous injection.

7. The method of claim 1, wherein the composition is administered in a dose of 6.75×10⁻⁸ mol PEG/kg.

8. The method of claim 1, wherein the composition is administered to the animal at least twice.

9. The method of claim 6, wherein a second dose of the composition is administered 14 days after the first administration.

10. The method of claim 1, wherein the animal produces anti-PEG antibodies within 28 days of the initial administration.

11. The method of claim 1, wherein the animal is a mouse.

12. An animal that produces anti-PEG antibodies generated by the method of claim 1.

13. A method for screening a PEG-containing product in vivo, the method comprising administering the product to the animal of claim 12.

14. The method of claim 13, wherein the screen is used to identify a potential adverse reaction to the product that occurs in the presence of anti-PEG antibodies.

15. The method of claim 13, wherein the screen is used to determine the immunogenicity of the product in the presence of anti-PEG antibodies.

16. The method of claim 13, wherein the product is a drug, an adjuvant, a nanoparticle, or a nanocarrier.

17. The method of claim 16, wherein the screen is used to determine the effective dose, pharmacokinetics, and/or biodistribution of the product in the presence of anti-PEG antibodies.

18. The method of claim 13, wherein the product is a medical device.

19. The method of claim 13, wherein the product is a food product, personal care product, or cleaning product.

20. The method of claim 14, wherein the animal is a mouse.
